(19) United States
(12) Patent Application Publication
(10) Pub. No.: US 2003/0162241 A1
(43) Pub. Date: Aug. 28, 2003

(54) Method for determining the concentration of carnitine in fluids

(76) Inventors: Fritz Pittner, Vienna (AT); Alfred Lohninger, Gaaden (AT); Thomas Schalkhammer, Kasten (AT); Christian Mayer, Delft (NL); Matthias Lepek, Vienna (AT); Claudia Micaela Dietrich-Ostry, Vienna (AT); Christian Vater, Vienna (AT); Alexander Piller, St. Andra/Wordern (AT)

Correspondence Address: Robert J Schneider, Chapman & Cutler, 111 West Monroe Street, Chicago, IL, 60603, US

(21) Appl. No.: 10/257,609

(22) PCT Filed: Apr. 6, 2001
(86) PCT No.: PCT/AT01/00100

(30) Foreign Application Priority Data
Apr. 17, 2000 (AT) A 679/2000

(51) Int. Cl. C12Q 1/26

(52) U.S. Cl. 435/25

(57) ABSTRACT

In a method of assay of the concentration of carnitine in biological fluids, the biological fluid containing carnitine is at least partially oxidized with a carnitine dehydrogenase (CDH) with the help of NAD⁺, whereupon the concentration of at least one reaction product, in particular the concentration of NADH, is determined.

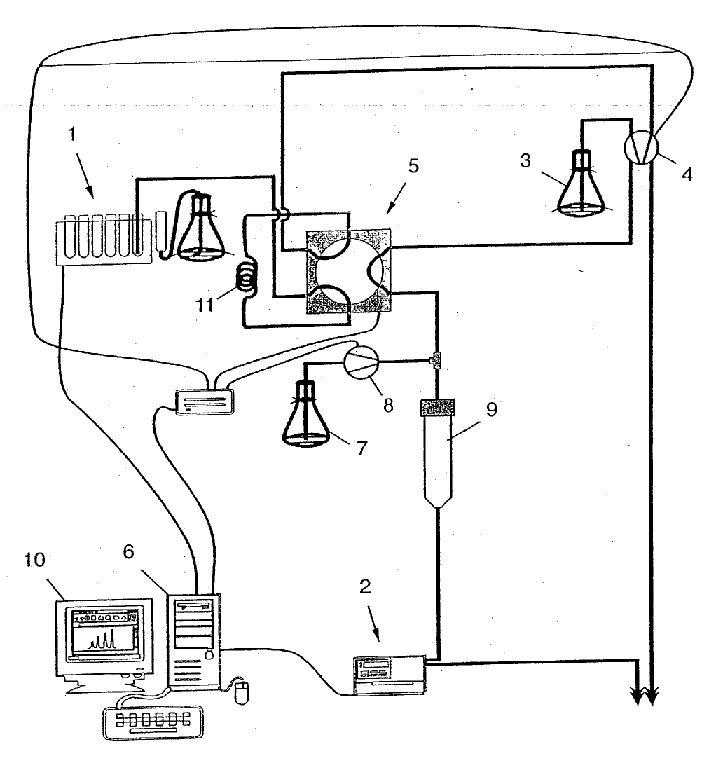

This invention relates to a method of assay of the carnitine concentration in biological fluids in which the carnitine-containing biological fluid is at least partially oxidized with a carnitine dehydrogenase (CDH) with the help of NAD⁺, whereupon the concentration of at least one reaction product, in particular the NADH concentration, is determined.

Numerous clinical studies of the carnitine metabolism suggest that secondary carnitine deficiency states are part of the clinical presentation of numerous deficiency diseases. Reduced carnitine levels are found in pregnant women, neonates, during infections and stress and in general when the metabolism has a high level of lipid turnover. So far there is no known simple method which can be carried out routinely and would permit a suitable analysis directly in the routine laboratory or at the specialist's office. Especially the concomitant monitoring during pregnancy could be made more efficient with such a simple method and would help lower the treatment costs in the area of neonatology and gynecology because of the improved diagnostics.

Carnitine (β-hydroxy-γ-N-trimethylammonium butanoic acid) is ubiquitous in nature and is characterized by a number of essential functions in the intermediate metabolism. In particular, the carnitine system transports activated short-chain, medium-chain and long-chain fatty acids throughout the entire cell area in the form of acyl carnitine esters. Long-chain fatty acids may enter the mitochondria only in the form of carnitine esters, and they are oxidized there to produce energy. Carnitine is also characterized by a regulatory role in controlling the ratio of acyl coenzyme A to free CoASH. Fatty acid residues are transferred from coenzyme A by specific acyl transferases.

Carnitine also plays an essential role in detoxification because intermediate metabolic products that are difficult to utilize accumulate in the cells as coenzyme A esters and can thus inhibit mitochondrial function. These compounds can be released by the cell as carnitine esters and eliminated through the kidneys. Finally, carnitine is especially important in activation of immunocompetent cells and for the stability of many membranes as well as for membrane synthesis and repair, in particular the erythrocyte membrane.

The principal reaction of carnitine with a carnitine dehydrogenase in which at least partial oxidation has been performed with the help of NAD⁺ and the reaction product, namely NADH, has subsequently been determined, has already been described in the literature. Reference is made here specifically to European Patent 437 373 A1. The specific parameters to be maintained for the assay can be derived in detail from the corresponding scientific publications in Clin. Chem. 36/12, 2072-2076 (1990) and it can be seen in particular that the fluorometric determination is performed with the required sensitivity in a known method only when a chemical amplifier is used. The reaction is performed with carnitine dehydrogenase and diaphorase, whereby resazurin is converted to resorufin to form a corresponding fluorescent pigment with which the desired sensitivity is achieved. The proposed device for fluorometric determination thus requires two reactors and can also be regarded as linear only in the narrow concentration range.

In addition to the increased expense for a second reactor, the additional reactants of course constitute an additional source of interference in the assay. French Patent 2 596 865 A1 proposes a method of metered addition of carnitine, here again using diaphorase and an electrochemical assay is proposed. Electrochemical assays are usually disturbed by a large number of redox active substances which include, for example, paracetamol, vitamin C and the like, which are also found in serum and therefore can have a negative effect on the assay.

The object of this invention is to create a rapid and simple method with which a quantitative assay of carnitine in the blood or serum is made possible with a reduced equipment complexity. To achieve this object, the process according to this invention consists essentially of injecting the sample into a carrier stream and passing it over CDH immobilized on a carrier on the way to a detector; before immobilization of CDH with BSA, blocking aminosilanized glass bodies with a controlled pore size (controlled pore glass beads, CPGs) at those locations where proteins can be physisorbed without activation; rinsing the CPGs with DTT solution before immobilization of the CDH; stabilizing the CDH by using a combination of DTT, NAD⁺ and EDTA and performing the assay of the NADH concentration by spectrophotometry or directly by fluorescence measurement. Due to the fact that the sample is merely injected into a carrier stream, a simple measurement method which has a low level of equipment complexity is proposed, whereby due to the special measures in conjunction with immobilization of CDH and preparation of the carrier, the sensitivity can be increased to the extent that a direct assay is possible without using chemical amplifiers or other auxiliary reagents. Specifically, the blockade with BSA performed before immobilization of CDH results in the immobilized enzyme being protected from oxidation and stabilized. BSA (bovine serum albumin) is a preparation of serum albumin which is free of protease and nuclease. Adding BSA results on the one hand in stabilization of the conformation of CDH and on the other hand both carboxyl groups and amino groups are made available by the BSA. Due to the combination of CPG-linker-BSA-CDH, the yield in immobilization can be increased, and thus more enzyme can be immobilized on the carrier, so that a simple measurement process is made possible. The DTT (dithiothreitol) which is also used in the process according to this invention acts as a stabilizing reagent for proteins carrying the SH groups. DTT reduces disulfide ridges and protects free SH groups. In the case of SH groups in the area of the active center, adding DTT acts mainly by improving the catalytic activity. The catalytic activity improved in this way makes it possible to achieve a direct assay in an especially rapid and simple method, e.g., by photometric assay of the NADH concentration. If a fluorescence measurement is performed as part of the process according to this invention, it can be performed directly with regard to the improvement and sensitivity achieved and without the assistance of additional reactions.

Preferably L-carnitine dehydrogenase from Agrobacterium sp. is used as the carnitine dehydrogenase, whereby this L-carnitine dehydrogenase oxidizes L-carnitine with the help of NAD⁺ to form dehydrocarnitine (β-oxo-γ-N-trimethylammonium butanoic acid). The reaction takes place according to the following equation:

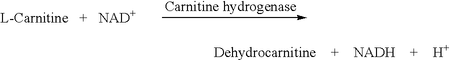

L-Carnitine dehydrogenase requires NAD⁺ as a coenzyme and has a molecular weight of 114 kD. The enzyme is highly specific for L-carnitine and NAD⁺ and it does not convert NADP⁺ or the closely related substances D-carnitine, D,L-4-amino-3-hydroxybutyric acid, 3-hydroxybutyric acid, choline, tartrate, malate, ethanol or isopropanol. A weak secondary activity (10%) was observed only in the case of D,L-carnitinamide.

There exist two different forms of the dehydrogenase, one with a high affinity for carnitine and one with a low affinity. The Kₘ values for the forms with a high or low affinity for L-carnitine are 0.29 and 6.1 mM (Vₘₐₓ 4.74 and 4.9 U/mol) and for NAD⁺ these values are 0.018 and 0.042 mM respectively. The enzyme is inhibited by detergents (cetyltrimethyl ammonium bromide and SDS) and by the heavy metal ions Ag⁺, Hg⁺, Co⁺, Hg²⁺, Zn²⁺, Cu²⁺, Mn²⁺, Ni²⁺, Pb²⁺ and by D-carnitine, p-chloromercuribenzoate, choline, hydroxylamine, borate and hydrazine hydrate and it is activated by magnesium and lithium. The active form of the enzyme is a dimer which has an isoelectric point at 5.4. The forward reaction has an optimum pH of 9 and the reverse reaction has an optimum pH of 7. The enzyme is thermally stable at temperatures up to 34° C. over along period of time and has an optimum temperature of 45° C. with a 100 second measurement time. The NADH formed in the above reaction can be measured photometrically or directly by fluorometric analysis in a simple manner, whereby a definite increase in absorbance is observed. In principle, acetyl carnitine can also be determined by the same method if such acetyl carnitine is not first reacted to yield carnitine. Carnitine transferases, esterases, or lipases or a basic hydrolysis may be used for the reaction of acetyl carnitine to carnitine. However, in any case the actual assay method consists of measuring the concentration of NADH from the reaction of carnitine with carnitine dehydrogenase (CDH).

The NADH concentration is determined according to this invention by spectrophotometry or by fluorescence measurement. The spectrophotometric assay can preferably take place at approximately 340 nm, whereas the direct fluorescence measurement is performed at wavelengths of 375 and 520 nm especially easily.

In comparison with the previously known, relatively complex assay method in which acetyl carnitine transferase, i.e., acetyl CoA-carnitine-O-acetyl transferase is used, the process according to this invention using CDH is characterized by a much lower substrate spectrum of the enzyme and avoidance of interference such as that observed in the case of acetyl transferases due to the presence of choline or tris-hydroxymethyl methylglycine. Thus, with respect to the specific reaction of carnitine, CDH is greatly superior to the specific assay of carnitine by the methods known in the past. The sensitivity can also be increased greatly because of the specific enzyme, and much lower costs are incurred. To reduce the continuous flushing of the system with NAD⁺ and the related costs, it is advantageous to proceed by diluting the sample with a measurement buffer, in particular Tris-HCl buffer containing NAD⁺, before the reaction with CDH, and then to bring it in contact with CDH.

Another advantage of the process according to this invention in addition to the higher specific sensitivity is also the relatively short reaction time so that reproducible signals can be achieved even after a short reaction time, even in the case of an incomplete reaction. Whereas in steady-state measurements, for example, sample solutions are passed over the enzyme until achieving a constant signal, a much faster measurement method is used according to this invention, and this method has become known as fluid [sic] injection analysis (FIA). Thus instead of an equilibrium measurement arrangement with a flow-through cell, an FIA measurement is selected in which the entire area is no longer used to determine the concentration but instead only the magnitude of the signal provides direct information regarding the concentration. To this end, the procedure according to this invention is such that the sample is injected into a carrier stream and is passed over CDH immobilized on a carrier on the way to a detector.

The use of CDH immobilized on a carrier represents another simplification and improvement in the reproducibility of the process according to this invention, the immobilization of CHD being accomplished by maintaining specific conditions. To this end, the procedure is to immobilize the CDH on an aminosilanized glass, whereby immobilization of CDH on aminosilanized glass is accomplished especially easily by means of 1-ethyl-3-(3-dimethylaminopropyl)carbodiimide (EDC). Aminosilanized glass bodies with a controlled pore size (CPGs) are used as the carriers which are blocked before immobilization of CDH by using BSA at the location where proteins can be physisorbed without activation. The immobilization is accomplished here by covalent bonding of the enzyme, where the EDC coupling selected has proven to be the most efficient method, which is easy to handle, of a mobilization on the surfaces of aminosilanized glass with a controlled pore size. The glass bodies with the immobilized enzyme can then be packed in columns and used in this way.

Carnitine dehydrogenase, like most enzymes, suffers a decline in activity over a period of time. To minimize the decline in activity of carnitine dehydrogenase, it is first necessary to ensure that the CDH is not already oxidized in immobilization, so the procedure is to wash the CPGs with DTT solution before immobilization of CDH. Dithiothreitol (DTT) has proven to be an excellent stabilizer for the enzyme CDH, and therefore the procedure followed in the process according to this invention is to stabilize the CDH by using a combination of DTT, NAD⁺ and EDTA.

The sensitivity of the photospectrometric measurement or the fluorescence measurement may be further increased by adding dyes to the measurement cell of the detector.

In addition to the stabilizers, DTT, NAD and EDTA (ethylenediamine tetraacetic acid) already described, the addition of protease inhibitors has also proven especially advantageous in stabilizing the activity of carnitine dehydrogenase.

In the assay methods according to this invention, a precisely defined amount of the liquid sample is injected into a continuously flowing carrier stream and transported to a detector using the FIA method. The equipment setup for performing this method is illustrated in this drawing. The sample goes first to an autosampler 1 and is injected into a continuously flowing carrier stream and then transported to a detector 2. On the way to the detector, the sample is mixed with a flow buffer 3, and a dye may also be added here. Use of a dye makes the measurement more efficient and more sensitive. The sample and the carrier stream are controlled by the pump 4 using injection valves 5 which are controlled fully automatically by software installed in a connected computer 6. After the NAD⁺ buffer is supplied, the sample goes from the container 7 through the pump 8 into a reactor 9 which contains the CDH immobilized on a carrier. In bioreactor 9 the carnitine is reacted and the reaction product that can be measured is formed. The NADH reaction product produces a signal in the spectrophotometer or detector 2 and this signal can be visualized in the form of a peak on monitor 10 of computer 6. In fully automatic processing of the signals, the carnitine content can be displayed here directly.

With a reaction module, i.e., with a packing of glass bodies with immobilized CDH, almost a thousand assays can be formed, which would correspond to a lifetime of approximately four months in a normal laboratory operation. The system can be recalibrated at any time and can be operated after brief training without any special knowledge.

The term flow injection analysis (FIA) describes a technique in which a precisely defined quantity of a liquid sample is injected into a continuously flowing carrier stream and is subsequently transported to a detector. The sample becomes mixed with the surrounding carrier solution on the way to the detector, with the carrier solution either serving only to transport the sample to the detector or it may also contain reagents for reaction of the analyte. Depending on the requirements, the dispersion of the samples zone can be controlled through various parameters, and in particular it can be influenced by the type and size of a mixing coil, which is labeled as 11 in the drawing, and by the diameter of the lines used and also by a change in the sample volume. In contrast with the continuous flow method, in flow injection analysis reproducible signals are obtained in the form of peaks although the mixing of the sample with the mobile buffer is not homogeneous and chemical reactions need not necessarily reach their equilibrium in the system process. The reproducibility of the FIA signals is based on the fact that the dwell time of the samples in the system and all the parameters that influence the dispersion of the sample are kept constant.

For the aminosilanization of glass bodies with a controlled pore size, aminopropylmethyl-diethoxysilane in toluene was used. The immobilization on such aminosilanized CPGs is preferably accomplished, as mentioned above, with 1-ethyl-3-(3-dimethylaminopropyl)carbodiimide (EDC) which converts a carboxyl group into a higher active ester intermediate, which can react with amines (amide bond) and thiols (thio ester bond) but also as a result of hydrolysis, it can disintegrate back into a carboxyl group and inactive EDC. Since CDH has proven to be relatively susceptible to oxidation, it is advantageous to largely eliminate the denaturing properties of the surface of the CPGs. To this end, it is suitable to block the CPGs with BSA (bovine serum albumin) and then rinse them with DTT (dithiothreitol). In blocking, all the physisorptive sites on the CPGs are occupied with BSA, whereas the subsequent rinsing with DTT washes out excess BSA as well as imparting reducing properties to each capillary. In addition, a protease inhibitor may also be used. After immobilization of CDH as part of the measurement method according to this invention, reproducible measurements in the μmol/L range can be achieved after a relatively short lead time with such pretreated CPGs.

1. A process for assay of the concentration of carnitine in biological fluids, in which the carnitine-containing biological fluid is at least partially oxidized with a carnitine dehydrogenase (CDH) with the help of NAD⁺, whereupon the concentration of at least one reaction product, including the concentration of NADH, is determined, characterized in that the sample is injected into a carrier stream and is passed over CDH immobilized on a carrier on the way to a detector, before immobilization of CDH, aminosilanized glass bodies with a controlled pore size (CPGs) are blocked with BSA at the sites where proteins can be physisorbed without activation; the CPGs are washed with DTT solution before immobilization of CDH; CDH is stabilized by using a combination of DTT, NAD⁺ and EDTA, and assay of the NADH concentration is performed by spectrophotometry or directly by fluorescence measurement.

2. The method according to claim 1, characterized in that before the reaction with CDH, the sample is diluted with a measurement buffer containing NAD⁺, in particular Tris-HCl buffer, and is brought in contact with CDH.

3. The method according to claim 1 or 2, characterized in that the immobilization of CDH on aminosilanized glass is performed by means of EDC.
